SEQUENCE LISTING (24 sequences)

SEQ ID NO: 1 - DNA, 20 bp, Artificial Sequence
  synthetic sequence
cagactcaatttagtgagcc

SEQ ID NO: 2 - DNA, 18 bp, Artificial Sequence
  synthetic sequence
ctgctggtccatgctgtt

SEQ ID NO: 3 - DNA, 21 bp, Artificial Sequence
  synthetic sequence
gaggaatgcagactcagtgga

SEQ ID NO: 4 - DNA, 21 bp, Artificial Sequence
  synthetic sequence
tagcttccactgctacctccg

SEQ ID NO: 5 - DNA, 20 bp, Artificial Sequence
  synthetic sequence
tcgtatgccacgctcatctg

SEQ ID NO: 6 - DNA, 20 bp, Artificial Sequence
  synthetic sequence
cggattgaattctgccaggt

SEQ ID NO: 7 - DNA, 20 bp, Artificial Sequence
  synthetic sequence
gctgcaaacccaatttagga

SEQ ID NO: 8 - DNA, 22 bp, Artificial Sequence
  synthetic sequence
tgctcatcaagttcagaaagga

SEQ ID NO: 9 - DNA, 20 bp, Artificial Sequence
  synthetic sequence
cctacaaagctgaggcctgt

SEQ ID NO: 10 - DNA, 20 bp, Artificial Sequence
  synthetic sequence
gaccctcctctcaatggtgc

SEQ ID NO: 11 - DNA, 24 bp, Artificial Sequence
  synthetic sequence
agcatcattaagctcatggaaaaa

SEQ ID NO: 12 - DNA, 20 bp, Artificial Sequence
  synthetic sequence
gtggactcaaagcatggcag

SEQ ID NO: 13 - DNA, 20 bp, Artificial Sequence
  synthetic sequence
aactgctggaggcgctatca

SEQ ID NO: 14 - DNA, 20 bp, Artificial Sequence
  synthetic sequence
tgtccttttcacgggtcact

SEQ ID NO: 15 - DNA, 20 bp, Artificial Sequence
  synthetic sequence
ctttgaactcagggtcacca

SEQ ID NO: 16 - DNA, 20 bp, Artificial Sequence
  synthetic sequence
tagtactgagccggatgcac

SEQ ID NO: 17 - DNA, 20 bp, Artificial Sequence
  synthetic sequence
tgcttcaggaaacataccga

SEQ ID NO: 18 - DNA, 20 bp, Artificial Sequence
  synthetic sequence
ctggagtgagctcctcatca

SEQ ID NO: 19 - DNA, 20 bp, Artificial Sequence
  synthetic sequence
tacacaggaatacaggactt

SEQ ID NO: 20 - DNA, 18 bp, Artificial Sequence
  synthetic sequence
ctcacaccactaccatct

SEQ ID NO: 21 - DNA, 20 bp, Artificial Sequence
  synthetic sequence
ctaggatgagcagctgtgga

SEQ ID NO: 22 - DNA, 20 bp, Artificial Sequence
  synthetic sequence
aagaatttgaagcgcacctt

SEQ ID NO: 23 - DNA, 21 bp, Artificial Sequence
  synthetic sequence
cttctaaccgaggtcgaaacg

SEQ ID NO: 24 - DNA, 22 bp, Artificial Sequence
  synthetic sequence
ggcattttggacaaakcgtcta

(19) United States
(12) Patent Application Publication
(10) Pub. No.: US 2021/0207081 A1
(43) Pub. Date: Jul. 8, 2021

(54) MATURE AIRWAY ORGANOIDS, METHODS OF MAKING AND USES THEREOF

(71) Applicants: THE UNIVERSITY OF HONG KONG, Hong Kong (CN); Koninklijke Nederlandse Akademie Van Wetenschappen, Amsterdam (NL)

(72) Inventors: Jie Zhou, Hong Kong (CN); Kwok Yung Yuen, Hong Kong (CN); Cun Li, Hong Kong (CN); Man Chun Chiu, Hong Kong (CN); Johannes Carolus Clevers, Amsterdam (NL)

(21) Appl. No.: 17/059,965

(22) PCT Filed: May 31, 2019
(86) PCT No.: PCT/CN2019/089613

§ 371 Date: Nov. 30, 2020

Related U.S. Application Data
(60) Provisional application No. 62/679,788, filed on Jun. 2, 2018.

(51) Int. Cl. C12N 5/00; C12N 7/00; G01N 33/50

(52) U.S. Cl. CPC C12N 5/0062; C12N 7/00; G01N 33/5082; C12N 2501/999; C12N 2501/30; C12N 2500/84; C12N 2513/00; C12N 2760/16011; C12N 2500/25; C12N 2501/11

(57) ABSTRACT

Provided are methods for generating 2D and 3D differentiated airway organoids, 2D and 3D differentiated airway organoids which are generated by the methods and uses for the 2D and 3D differentiated airway organoids.

### FIELD OF THE INVENTION

The invention is generally directed to airway organoids, particularly differentiated airway organoids, methods of making and using, particularly for influenza virus research.

### BACKGROUND OF THE INVENTION

Influenza A viruses (IAVs) can infect a diversity of avian and mammalian species including humans, and have the remarkable capacity to evolve and adapt to new hosts (1). The segmented RNA genomes of IAVs and the low fidelity of RNA polymerase allow for antigenic shift and drift, which drive this evolution. Thus, novel viruses from birds and pigs can cross the species barrier and infect humans, leading to sporadic infections, epidemics and even pandemics (Klenk, Cell Host Microbe 15(6):653-654 (2014); To, et al., Lancet 381(9881):1916-1925 (2013)). Despite the tremendous progress made in virology and epidemiology, it remains unpredictable which subtype or strain of IAV will cause the next outbreak. A novel reassortant H7N9 influenza virus from poultry has led to recurrent outbreaks of human infections in China since 2013 (To, et al., Lancet 381(9881):1916-1925 (2013)), Chen, et al., Lancet 381(9881):1916-1925 (2013)). According to a World Organization report more than 1500 laboratory-confirmed cases of H7N9 human infections were reported by October 2017, with a case-fatality rate higher than 35%. In 2009, the first influenza pandemic of the 21^{st} century was caused by a novel pandemic H1N1 (H1N1pdm), which originated via multiple reassortment of “classical” swine H1N1 virus with human H3N2 virus, avian virus and avian-like swine virus (AVIT, et al., N Engl J Med 360(25):2605-2615 (2009)). While swine viruses only sporadically infect humans, this novel strain of swine-derived H1N1pdm virus can establish sustained human-to-human transmission and has been circulating globally as a seasonal virus strain since then. Proteolytic cleavage of viral glycoprotein hemagglutinin (HA) is essential for IAV to acquire infectivity since only the cleaved HA molecule mediates the membrane fusion between virus and host cell, a process required for the initiation of infection. HA proteins of low pathogenic avian IAVs and human IAVs carry a single basic amino acid arginine at the cleavage site (Bottcher E, et al., J Virol 80(19):9896-9898 (2006); Bosch, et al., Virology 113(2):725-735 (1981)), recognized by trypsin-like serine proteases. Productive infection of these viruses in human airway thus requires serine proteases like TMPRSS2, TMPRSS4, HAT etc. (Bottcher-Friebertshauser et al., Pathog Dis 69(2):87-100 (2013). However, HA proteins of high pathogenic avian viruses, such as H5N1, contain a polybasic cleavage site that is activated by ubiquitously expressed proteases.

Current in vitro models for studying influenza infection in human respiratory tract involve short-term cultures of human lung explant and primary airway epithelial cells. Human lung explants are not readily available on a routine basis. In addition, rapid deterioration of primary tissue in infection experiments is a major problem. Under air-liquid interface conditions, basal cells isolated from human airway can polarize and undergo mucociliary differentiation. Yet, this capacity is lost within 2-3 passages (Butler, et al., Am J Respir Crit Care Med 194(2):156-168 (2016)). Collectively, these primary tissues and cells barely constitute a convenient, reproducible model to study human respiratory pathogens. Although various cell lines, e.g. A549 and MDCK, have commonly been used to propagate influenza viruses and to study virology, they poorly recapitulate the histology of human airway epithelium. In addition, due to the low serine protease activity, most cell lines do not support the growth of the influenza viruses with monobasic HA cleavage site unless the culture medium is supplemented the exogenous serine protease, trypsin treated with N-tosyl-L-phenylalanine chloromethyl ketone (TPCK). Thus, a biologically-relevant, reproducible, and readily-available in vitro model remains desperately needed for studying biology and pathology of the human respiratory tract.

Recent advances in stem cell biology have allowed the in vitro growth of 3 dimensional (3D) organoids that recapitulate essential attributes of their counterpart-organs in vivo. These organoids can be grown from pluripotent stem cells (PSC) or tissue-resident adult stem cells (ASC) (Clevers, et al., Cell 165(7):1586-1597 (2016)). ASC-derived organoids consist exclusively of epithelial cells and can be generated from a variety of human organs, the first being the human gut (Sato, et al., Gastroenterology 141(5):1762-1772 (2011)). These human intestinal organoids represent the first model for in vitro propagation of Norovirus and has allowed the study of other viruses (Ettayebi, et al., Science, 353:1387-1393 (2016); Zhou, et al., Sci. Adv. 3(11);eaao4966 (2017)). ASC-derived lung organoids have also been described (WO2016/083613).

Of note, protocols have also been established to generate lung organoids from human PSCs, embryonic lung (Chen, et al., Nat Cell Biol 19(5):542-549, (2017); Nikolic, et al., Elife 6: e26575 (2017)), embryonic stem cells and induced pluripotent stem cells (iPSC) (Konishi, et al., Stem Cell Reports, 6(1):18-25 (2016)).

However, there is still a need for improved methods of generating in vitro cellular systems that recapitulate the histology and functionality of mature (differentiated) human airway epithelium, for example, for use in modelling infection, particularly influenza infection. There is, in particular, a need for improved methods of differentiating ASC-derived lung organoids. Such methods would be advantageous because they do not rely on induced pluripotent stem cells, embryonic stem cells or embryonic lung. Therefore, it is the object of the present invention to provide a method of generating an in vitro cellular system that recapitulates the histology and functionality of mature human airway epithelium for use in modelling diseases, for example, influenza infection.

It is another object of the present invention to provide a method of differentiating lung organoids, preferably wherein said method does not rely on induced pluripotent stem cells, embryonic stem cells or embryonic lung.

It is another object of the present invention to provide improved in vitro differentiated lung organoids that recapitulate the histology of human airway epithelium.

It is yet another object of the present invention to provide methods for studying the biology and pathology of the human airway epithelium.

### SUMMARY OF THE INVENTION

Methods for obtaining a population of differentiated airway epithelial cells, differentiated airway epithelial cells generated by the disclosed methods and uses for the differentiated airway epithelial cells are provided.

In particular, methods for generating two-dimensional (2D) and three-dimensional (3D) differentiated airway organoids, differentiated 2D and 3D airway organoids generated by the disclosed methods, and uses for the 2D and 3D airway organoids are provided.

The methods of generating differentiated airway organoids include obtaining a lung tissue sample from a subject, obtaining dissociated cells from the lung sample, culturing the dissociated cells in a two phase process (a) organoid formation phase and (b) organoid maturation phase. The organoid formation phase involves culturing the dissociated cells in an airway organoid (AO) medium for a period of time sufficient to form an airway organoid. The organoid maturation phase involves culturing the airway organoids from the formation phase in proximal differentiation (PD) medium, for a period of time effective to improve morphology and differentiation of cells in the organoid. Criteria indicating an improvement in morphology and differentiation include for example, an increase in the percentage of ciliated cell number following culture in PD medium. 2D and 3D airway organoids obtained by a combination of AO and PD culture are referred to herein as proximal differentiated airway organoids (or “PD-organoids”). 2D differentiated organoids are obtained by a method that comprises dissociating the 3D airway organoids into a single cell suspension, and seeding the cells in transwell inserts, before culturing the cells in PD medium, preferably for a period of time effective for formation of an intact epithelial barrier. This can be measured by trans-epithelial electronic resistance and a dextran penetration assay.

The disclosed PD-organoids include a combination of basal cells, goblet cells, club cells and enriched ciliated cells, and accordingly, express one or more markers selected from the group consisting of ciliated cell markers (FOXJ1 and SNTN), basal cell markers (P63, CK5); goblet cell marker (MUC5AC) and increased serine proteases including TMPRSS2, TMPRSS4, TMPRSS11D (HAT) and Matriptase. In one preferred embodiment, PD organoids are disclosed which include readily discernible ciliated cells at a percentage greater than 10%, preferably, greater than 20%. For example, ciliated cells can make up at least 40% of the cells in the organoid, at day 14, preferably, day 16 post PD cell culture. 2D PD airway monolayers are provided, with an intact epithelial barrier to simulate the real human airway epithelium and model the natural mode of pathogen exposure to the human airway.

Also disclosed are methods to evaluate the biology and pathology of human airway epithelium for example, to assess the infectivity of an emerging influenza virus, such as H7N9.

### BRIEF DESCRIPTION OF THE DRAWINGS

FIGS. 1A-1C show viral loads in airway organoids inoculated with H1N1pdm, H5N1 and H7N9/Ah. The airway organoids were inoculated with H1N1pdm, H5N1 and H7N9/Ah at an MOI of 0.01. The infected organoids (cell lysate) and supernatants were harvested at the indicated hours to detect the viral loads. Supernatant samples were used for viral titration. Data showing mean±SD of triplicated samples.

FIG. 2 shows the diameters of individual organoids. The images of organoids cultured in PD medium and AO medium are used to measure the diameters of individual organoids (n=300) using ImageJ. Student's T test was used for data analysis. ***, P<0.005.

FIGS. 3A-D show characterization of the differentiation status of airway organoids. (FIGS. 3A and 3B). Fold changes in expression levels of cell type markers (FIG. 3A) and serine proteases (FIG. 3B) in the organoids cultured in PD medium versus those in AO medium at the indicated day. Data show mean and SD of two lines of organoids. FIG. 3C shows the percentages of individual cell types in the organoids cultured in PD medium and AO medium. The representative histograms of one organoid line are shown. FIG. 3D shows fold changes in positive cell percentages in the organoids cultured in PD medium versus those in AO medium.

FIGS. 4A-C. Influenza virus infection in the 3D PD airway organoids. The 3D PD airway organoids were inoculated with H7N9/Ah and H7N2 virus at an MOI of 0.01. The infected organoids (cell lysate) and supernatants were harvested at the indicated hours to detect the viral loads (FIGS. 4A and 4B). Supernatant samples were used for viral titration (FIG. 4C). Data showing mean±SD of triplicated samples in one representative experiment repeated 3 times.

FIGS. 5A-B show formation of epithelial barrier in 2D monolayers of differentiated airway organoids in transwell culture. The 3D airway organoids were dissociated into single cells, seeded in transwell inserts and cultured in AO medium. At day 2, AO medium was replaced with PD medium. (A) Trans-epithelial electronic resistance (TEER) was measured at the indicated day post seeding. Data show the cell-specific TEER (mean±SD) of 2D monolayers in 10 inserts. (B) At day 10 after transwell culture, Fluorescein isothiocyanate-dextran (MW10k) was added in the medium of upper chamber and incubated for 4 hours. The medium in the upper and bottom chamber were collected and applied to fluorescence assay. Dextran blockage index refers to the fluorescence intensity of the medium in the upper chamber versus that in the bottom chamber. Data represent mean and SD of 10 inserts seeded with 2D airway organoids and those in two blank inserts. FIGS. 5C and 5D show replication capacity of influenza viruses in established 2D differentiated airway organoids. 2D PD airway organoids were inoculated in duplicate with H7N9/Ah, H7N2 as well as H1N1pdm, H1N1sw at an MOI of 0.001. The cell-free media were harvested from apical and basolateral chambers at the indicated hours post infection (hpi) for viral titration.

### DETAILED DESCRIPTION OF THE INVENTION

### I. Definitions

A “base media,” as used herein, refers to a basal salt nutrient or an aqueous solution of salts and other elements that provide cells with water and certain bulk inorganic ions essential for normal cell metabolism and maintains intra-cellular and/or extra-cellular osmotic balance.

An ErbB3/4 ligand is herein defined as a ligand that is capable of binding to ErbB3 and/or ErB4.

The term “Induced pluripotent stem cell” (iPSC), as used herein, is a type of pluripotent stem cell artificially derived from a non-pluripotent cell.

“Media” or “culture media” as used herein refers to an aqueous based solution that is provided for the growth, viability, or storage of cells used in carrying out the present invention. A media or culture media may be natural or artificial. A media or culture media may include a base media and may be supplemented with nutrients (e.g., salts, amino acids, vitamins, trace elements, antioxidants) to promote the desired cellular activity, such as cell viability, growth, proliferation, and/or differentiation of the cells cultured in the media.

“Organoid” as used herein refers to an artificial, in vitro construct derived from adult stem cells created to mimic or resemble the functionality and/or histological structure of an organ or portion thereof.

The term “pluripotency” (or pluripotent), as used herein refers to a stem cell that has the potential to differentiate into any of the three germ layers: endoderm (for example, interior stomach lining, gastrointestinal tract, the lungs), mesoderm (for example, muscle, bone, blood, urogenital), or ectoderm (for example, epidermal tissues and nervous system).

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Compositions

2D and 3D airway organoids are provided, differentiated by culture of cells obtained from lung tissue, in AO culture medium followed by culture in PD cell culture medium. An organoid is a cellular cluster derived from stem cells or primary tissues and exhibits endogenous organ architecture. See, e.g., Cantrell and Kuo, Genome Medicine 7:32-34 (2015). Organoids differ from naturally occurring in vivo tissues and from ex vivo tissue explants because they are derived from expansion of epithelial tissue cells only.

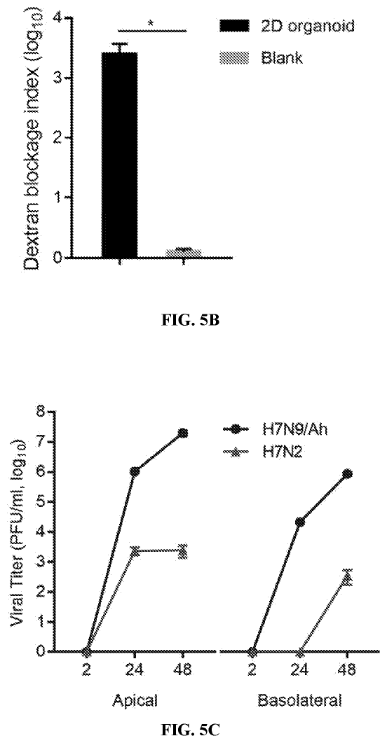

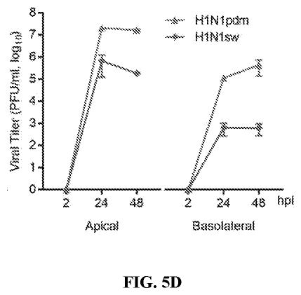

The disclosed 3D and 2D differentiated airway organoids support active replication of human infective H7N9/Ah and H1N1pdm. In contrast, the H7N2 virus, which has been temporally and spatially co-circulating with H7N9 viruses in domestic poultry and contains the similar internal genes as H7N9 viruses, replicated much less efficiently in both models. Similarly, the swine H1N1 isolate showed a lower growth capacity than its counterpart of human-adapted H1N1pdm (FIG. 5B). Thus, these PD airway organoids discriminate human infective viruses from poorly infective viruses.

In particularly preferred embodiments cells the disclosed 2D and 3D organoids do not recombinantly express of Oct3/4, Sox2, Klf4, c-Myc, L-MYC, LIN28, shRNA for TP53 or combinations thereof, i.e., the 2D and 3D organoids do not include cells genetically engineered to Oct3/4, Sox2, Klf4, c-Myc, L-MYC, LIN28, shRNA for TP53 or combinations thereof.

A. 3D PD-Airway Organoids

In vitro 3D airway organoids are disclosed. The airway organoids are 3D cysts lined by polarized epithelium. The disclosed airway organoids include a combination of basal cells, ciliated cells, goblet cells, and club cells, and accordingly, express one or more markers selected from the group consisting of ciliated cell markers (FOXJ1 and SNTN), basal cell markers (P63, CK5); goblet cell marker (MUC5AC) and serine proteases including TMPRSS2, TMPRSS4, TMPRSS11D (HAT) and Matriptase. Ciliary beating plays essential roles in human airway biology and pathology, and 50%-80% of airway epithelial cells are ciliated (Yaghi, et al., Cells, 5(4): pii:E402016)). The data in this application demonstrates that the ability to obtain airway organoids with a ciliated cell population that approaches physiological levels (i.e., more than 40% of the total population of organoid cells), depends on the cell culture medium selection (i.e. the factors used to supplement basal medium) as well as the cell culture protocol used to culture cells obtained from lung tissue i.e., timing of when cells are exposed cells to the combination of factors used to supplement basal medium). In a particularly preferred embodiment, the 3D PD-airway organoids contain no type I and type II alveolar epithelial cells in contrast to whole lung tissue, and the cilia on the PD-organoids beat synchronously. The disclosed organoids, generated from in vitro culture using a combination of AO and PD culture medium (PD-organoids) show improved expression of these markers, when compared to airway organoids generated from in vitro culture in AO culture medium alone (AO-organoids) for the same length of time. Criteria indicating an improvement in morphology and differentiation include for example, an increase in the percentage of ciliated cells following culture in PD medium. When compared to 3D AO-organoids, PD-organoids contain an increased level of ciliated and goblet cells, for example, a 2 fold, to 100 fold increase. In one preferred embodiment, PD organoids are disclosed which include ciliated cells with a near-physiological abundance at a percentage greater than 10%, preferably, greater than 20%. For example, ciliated cells can make up at least 40% of the cells in the organoid, at day 16 post PD cell culture. Thus, the PD organoids contain about 40% ciliated cells, preferably, between 40 and 50% ciliated cells at day 16 post PD medium cell culture. Meanwhile 3D PD-organoids contain a decreased level of club cells when compared to 3D AO-organoids.

PD-organoids show reduced expression of Club cell markers (CC10, SCGB3A2) compared to AO-organoids.

B. 2D Differentiated Airway Organoids

2D PD airway monolayers are provided, with an intact epithelial barrier to allow exclusive apical exposure. The presence of an intact epithelial barrier is determined for example using Transepithelial electrical resistance (TEER). Stabilization of TEER measurement shows formation of an intact barrier as shown for Example in FIG. 5A (shows stabilization of TEER at day 6). The electrical resistance of a cellular monolayer, measured in ohms, is a quantitative measure of the barrier integrity. Other methods of measuring monolayer integrity are known in the art. Reviewed in Elbrecht, et al., J. Rare Disease and Treat. 1(3):46-52 (2016); benson, et al., Fluids Barriers CAN, 10:5 (2013).

A limitation of 3D organoids for studying microbial infections is the inaccessibility of apical surface to pathogens since most organoids are orientated inwards, while receptors for most respiratory viruses are distributed in the apical surface. For virus inoculation, organoids have to be sheared to enable sufficient apical exposure to the virus inoculum (Drumond, et al., P.N.A.S., 114(7):1672 2677 (2017)).

The disclosed 2D PD organoids include an apical side and a basolateral side. Cells in the 2D organoid include a combination of basal cells, ciliated cells, goblet cells, and club cells, and accordingly, express one or more markers selected from the group consisting of ciliated cell markers (FOXJ1 and SNTN), basal cell markers (P63, CK5); goblet cell marker (MUCSAC) and serine proteases including TMPRSS2, TMPRSS4, TMPRSS11D (HAT) and Matriptase. In a particularly preferred embodiment, the 3D PD-airway organoids contain no type I and type II alveolar epithelial cells.

### III. Methods of Making Airway Organoids

The disclosed methods outline steps for culturing cells obtained from lung tissue to generate 3D organoids.

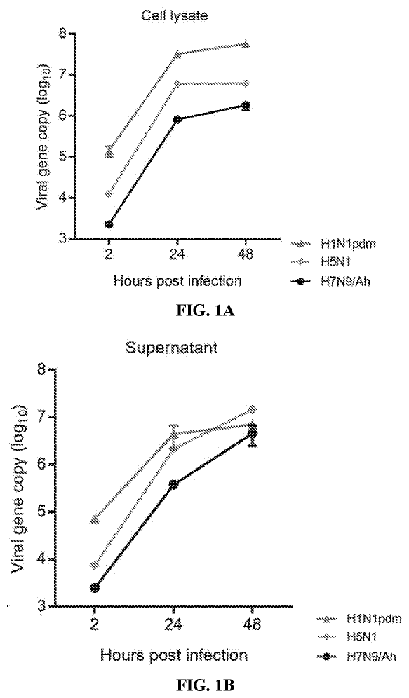

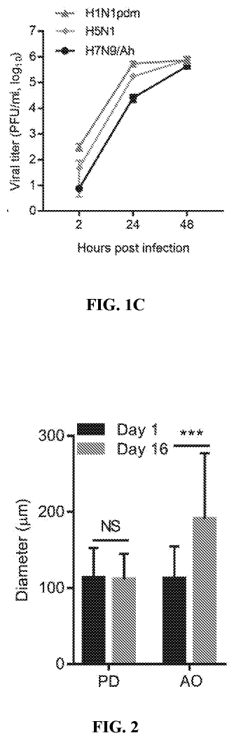

Airway adult stem cell (ASC)-derived organoids disclosed herein, once established, can be expanded indefinitely while displaying remarkable phenotypic and genotype stability. They thus overcome the reproducibility and availability limitations of the current in vitro model systems. Several lines of airway organoids were obtained from small pieces of normal lung tissue adjacent to the diseased tissue from patients undergoing surgical resection for clinical conditions. These airway organoids, 3D cysts lined by polarized epithelium, include the four major types of airway epithelial cells, i.e. ciliated cell (ACCTUB+ or FOXJ1+), basal cell (P63+), goblet cell (MUC5AC+), and Club cell (CC10+) (FIG. 1A). The cell culture media used to generate the airway organoids in some preferred embodiments does not include BMP (bone morphogenic protein) 4. In one preferred embodiment, generating a line of 3D organoids from primary lung tissues in AO culture medium disclosed herein takes preferably between one and four weeks, more preferably, between 2 and 3 weeks.

A. 3D PD-Airway Organoids

One embodiment provides a method of making an organoid from a mammalian tissue in vitro comprising: (a) obtaining a lung tissue sample from a subject, (b) isolating cells from the mammalian tissue to provide isolated cells by subjecting the tissue sample into single cells; (c) culturing the cells in an airway organoid (AO) culture medium for at least one to four weeks, preferably between 2 and 3 weeks to generate 3D airway organoids. The established 3D airway organoids can be maintained in AO medium and passaged every two to three weeks. (d) and preparing (adjusting) the established 3D airway organoids to an appropriate state (e) culturing the 3D airway organoids in differentiation medium, preferably a proximal differentiation medium (PD), for a time sufficient to produce differentiated airway organoids. In step (d),the 3D airway organoids are split and maintained in AO medium for at least 2 to 16 days, for example 2 days, 3 days, 4 days, 5 days, 6 days, 7 days, 8 days, 9 days, 10 days, 11 days, 12 days, 13 days, 14 days, 15 days or 16 days. Steps (c) and (d) are preferably three dimensional (3D) cell culture, as opposed to 2D cell culture. While the 2D culture usually grows cells into a monolayer on glass or, more commonly, tissue culture polystyrene plastic flasks, 3D cell cultures grow cells into 3D aggregates/spheroids using a scaffold/matrix. Commonly used scaffold/matrix materials include biologically derived scaffold systems and synthetic-based materials.

In some preferred embodiment, the method is performed with a commercially using extracellular matrix. In some preferred embodiment, the method is performed with a commercially available extracellular matrix such as MATRIGEL™ (growth Factor Reduced Basement Membrane Matrix). Other extracellular matrices (ECM) are known in the art for culturing cells. A preferred ECM for use in a method of the invention includes at least two distinct glycoproteins, such as two different types of collagen or a collagen and laminin. In some preferred embodiment, the method is performed with a commercially available extracellular matrix such as MATRIGEL™ (growth Factor Reduced Basement Membrane Matrix), which comprises laminin, entactin, and collagen IV. In general, an extracellular matrix comprises laminin, entactin, and collagen. In a preferred embodiment, the method is performed using a 3-dimensional culture device (chamber) that mimics an in vivo environment for the culturing of the cells, where preferably the extracellular matrix is formed inside a plate that is capable of inducing the proliferation of stem cells under hypoxic conditions. Such 3-dimensional devices are known in the art. Other commercially available products include Cultrex® basement membrane extract (BME; Trevigen), and hyaluronic acid are commonly used biologically derived matrixes. Polyethylene glycol (PEG), polyvinyl alcohol (PVA), polylactide-co-glycolide (PLG), and polycaprolactone (PLA) are common materials used to form synthetic scaffolds. Scaffold-free 3D cell spheroids can be generated in suspensions by the forced floating method, the hanging drop method, or agitation-based approaches. Edmondson, et al., Assay Drug. Dev. Technol., 12(4):207-218 (2014). For example, the isolated cells are embedding in 60% MATRIGEL™ and seeded in a suspension culture plate prior to culture in the (AO) medium.

In still another preferred embodiment, the AO culture medium step does not include cells expressing Oct4 and/or are not genetically engineered to express one or more markers of pluripotency i.e., the cells iPSC, for example, adult cells induced to pluripotency by expression of Oct3/4, Sox2, Klf4, c-Myc, L-MYC, LIN28, shRNA for TP53 or combinations thereof, or embryonic stem cells, for example, H9 hESCs (Thomson et al., Science 282:1145-1147 (1998)), 201B7 (Takahashi et al., Cell, 131(5):861-72 (2007)), 585A1 or 604A1 hiPSCs (Okita et al., Stem Cells, 31(3):458-66 (2013)).

(i) Sources for Airway Organoids

The disclosed organoids can be cultured from a tissue sample preferably a lung tissue sample obtained from a mammal, such as any mammal (e.g., bovine, ovine, porcine, canine, feline, equine, primate), preferably a human.

In a preferred embodiment, the lung tissue is not obtained from an embryonic human lung, and is preferably obtained from non-embryonic lungs for example, juvenile or adult lungs, preferably, adult lung.

In one embodiment, single cells are obtained from a tissue sample using a combination of steps that result in single cells. The tissue sample size can range in size from 0.1 cm to 10 cm, for example, between 0.5 and 5 cm, in some preferred embodiments between 0.5 and 1.0 cm in size. Cells may be isolated by disaggregating an appropriate organ or tissue that is to serve as the cell source using techniques known to those skilled in the art. For example, the tissue or organ can be disaggregated mechanically and treated with digestive enzymes and/or chelating agents to release the cells, to form a suspension of individual cells. Enzymatic dissociation can be accomplished by mincing the tissue and treating the minced tissue with one or more enzymes such as trypsin, chymotrypsin, collagenase, elastase, and/or hyaluronidase, DNase, pronase, dispase etc.

In a preferred embodiment, single cells are obtained from the lung tissue sample by mincing a lung tissue sample obtained from a subject, digesting with collagenase for 1 to two hours at 37° C., followed by shearing using glass Pasteur pipette and straining over a filter, for example, a 100 μm cell strainer.

In another preferred embodiment adult stem cells are obtained from lung tissue sample by selecting for cells expressing the Lgr5 and/or receptor, which belong to the large G protein-coupled receptor (GPCR) superfamily. One embodiment includes preparing a cell suspension from lung tissue, contacting the cell suspension with cells expressing the Lgr5 and/or receptor, isolating the Lgr5 and/or 6 binding compound, and isolating the stem cells from the binding compound. Examples of Lgr5 and/or Lgr6 binding compounds include antibodies, such as monoclonal antibodies, that specifically recognize and bind to the extracellular domain of either Lgr5 or Lgr6. Using such an antibody, Lgr5 and/or Lgr6-expressing stem cells can be isolated using methods known in the art, for example, with the aid of magnetic beads or through fluorescence-activated cell sorting.

In one preferred embodiment the disclosed method does not include the step of selecting for cells expressing any markers, for example, the Lgr5 and/or receptor, using Lgr5 and/or Lgr6 binding compounds or biomarkers for lung disease, such as CPM (carboxypeptidase M) (Dragavic, et al., Am. J. Respir. Crit Care Med., 152:760-764 (1995). This embodiment contemplates a method of generating airway organoids, that does not include enriching the population of starting cells based on surface marker expression

Isolated cells are further cultured as discussed herein. A preferred cell culture medium is a defined synthetic medium, buffered at a pH of 7.4 (preferably between 7.2 and 7.6 or at least 7.2 and not higher than 7.6) with a carbonate-based buffer, while the cells are cultured in an atmosphere comprising between 5% and 10% CO₂, or at least 5% and not more than 10% CO₂, preferably 5% CO₂.

(ii) AO Culture Medium

The cells are cultured in supplemented basal cell culture media. In some embodiments, a base media may include at least one carbohydrate as an energy source and/or a buffering system to maintain the medium within the physiological pH range. Examples of commercially available base media may include, but are not limited to, phosphate buffered saline (PBS), Dulbecco's Modified Eagle's Medium (DMEM), Minimal Essential Medium (MEM), Basal Medium Eagle (BME), Roswell Park Memorial Institute Medium (RPMI) 1640, MCDB 131, Click's medium, McCoy's 5 A Medium, Medium 199, William's Medium E, insect media such as Grace's medium, Ham's Nutrient mixture F-10 (Ham's F-10), Ham's F-12, a-Minimal Essential Medium (aMEM), Glasgow's Minimal Essential Medium (G-MEM) and Iscove's Modified Dulbecco's Medium. A preferred basal cell culture medium is selected from DMEM/F12 and RPMI 1640. In a further preferred embodiment, Advanced DMEM/F12 or Advanced RPMI is used, which is optimized for serum free culture and already includes insulin. In this case, the Advanced DMEM/F 12 or Advanced RPMI medium is preferably supplemented with glutamine and Penicillin/streptomycin. In preferred embodiments, the basal medium comprises Gastrin. In some embodiments, the basal medium also comprises NAc and/or B27.

In some embodiments an AO medium as described in WO2016/083613 can be used. In a particularly preferred embodiment, an AO culture medium (Table 1) is used, which is supplemented base media suitable to maintain airway organoids in culture.

The AO culture medium is base medium supplemented with agents such as Rspondin (a Wnt agonist), a BMP inhibitor, a TGF-beta inhibitor, a fibroblast growth factor (FGF) and Nicotinamide.

In some embodiments, the supplemented basal culture medium used to culture cells dissociated from a tissue sample does not include a GSK3 inhibitor, for example CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile). Known GSK-inhibitors comprise small-interfering RNAs, 6-Bromoindirubin-30-acetoxime.

A preferred AO medium is shown in Table 1.

TABLE 1
Composition of human airway organoid (AO) medium.

| | | | Working | |
|---|---|---|---|---|
| Reagents | Company | Catalog No. | concentration | |
| Advanced DMEM/F12 | Invitrogen | 12634010 | n/a | |
| HEPES | Invitrogen | 15630-056 | 1% | |
| GlutaMAX | Invitrogen | 35050061 | 1% | |
| Penicillin-Streptomycin | Invitrogen | 15140-122 | 1% | |
| Rspondin1* (conditioned | n/a | n/a | 10% | |
| medium) | | | | |
| Noggin* (conditioned | n/a | n/a | 10% | |
| medium) | | | | |
| B27 supplement | Invitrogen | 17504-044 | 2% | |
| N-acetylcysteine | Sigma | A9165 | 1.25 | mM |
| Nicotinamide | Sigma | N0636 | 10 | mM |
| Y-27632 | Tocris | 1254 | 5 | μM |
| A8301 | Tocris | 2939 | 500 | nM |
| SB202190 | Sigma | S7067 | 1 | μM |
| FGF-7 | Peprotech | 100-19 | 5 | ng/ml |
| FGF-10 | Peprotech | 100-26 | 20 | ng/ml |
| Primocin | InvivoGen | ant-pm-1 | 100 | μg/ml |
| Heregulin beta-1 | Peprotech | 100-03 | 5 | nM |

*Conditioned media were produced from stable cell lines for production of R-spondin1 and Noggin.

The AO medium incudes a BMP inhibitor. BMP inhibitor is defined as an agent that binds to a BMP molecule to form a complex wherein the BMP activity is neutralized, for example by preventing or inhibiting the binding of the BMP molecule to a BMP receptor. Alternatively, the inhibitor is an agent that acts as an antagonist or reverse agonist. BMP-binding proteins that can be used in the disclosed methods include, but are not limited to Noggin (Peprotech), Chordin and chordin-like proteins (R&D systems) comprising chordin domains, Follistatin and follistatin-related proteins (R&D systems) comprising a follistatin domain, DAN and DAN-like proteins (R&D systems) comprising a DAN cysteine-knot domain, sclerostin/SOST (R&D systems), decorin (R&D systems), and alpha-2 macroglobulin (R&D systems). Most preferred BMP inhibitor is Noggin. Noggin is preferably added to the basal culture medium at a concentration of at least about 10%.

The AO medium incudes a WNT agonist. Wnt agonists include the R-spondin family of secreted proteins, which is include of 4 members (R-spondin 1 (NU206, Nuvelo, San Carlos, Calif.), R-spondin 2 ((R&D systems), R-spondin 3, and R-spondin-4); and Norrin. In a preferred embodiment, a Wnt agonist is selected from the group consisting of: R-spondin, Wnt-3a and Wnt-6. Preferred concentrations for the Wnt agonist are about 10% for R-spondin and approximately 100 ng/ml or 100 ng/ml for WNt-3a. In some preferred embodiments, the WNT agonist is not a GSK inhibitor.

SB 202190 (4-(4-Fluorophenyl)-2-(4-hydroxyphenyl)-5-(4-pyridyl)-1H-imidazole) is a highly selective, potent and cell permeable inhibitor of p38 MAP kinase. SB 202190 binds within the ATP pocket of the active kinase (K_{d}=38 nM, as measured in recombinant human p38), and selectively inhibits the p38a and β isoforms. Other useful p38 MAPK inhibitors include, but are not limited SB203580 (4-[5-(4-Fluorophenyl)-2-[4-(methylsulfonyl)phenyl]-1H-imidazol-4-yl]pyridine); SB 203580 hydrochloride (4-[5-(4-Fluorophenyl)-2-[4-(methylsulphonyl)phenyl]-1H-imidazol-4-yl]pyridine hydrochloride); SB202190 (4-[4-(4-Fluorophenyl)-5-(4-pyridinyl)-1H-imidazol-2-yl]phenol); DBM 1285 dihydrochloride (N-Cyclopropyl-4-[4-(4-fluorophenyl)-2-(4-piperidinyl)-5-thiazolyl]-2-pyrimidinamine dihydrochloride); SB 239063 (trans-4-[4-(4-Fluorophenyl)-5-(2-methoxy-4-pyrimidinyl)-1H-imidazol-1-yl]cyclohexanol); SKF 86002 dihydrochloride (6-(4-Fluorophenyl)-2,3-dihydro-5-(4-pyridinyl)imidazo[2,1-b]thiazole dihydrochloride).

A8301 (3-(6-Methyl-2-pyridinyl)-N-phenyl-4-(4-quinolinyl)-1.H-pyrazole-1-carbothioamide) is potent inhibitor of TGF-β type I receptor ALK5 kinase, type I activin/nodal receptor ALK4 and type I nodal receptor ALK7, A83-01 may be added to the culture medium at a concentration of between 10 nM and 10 uM, or between 20 nM and 5 uM, or between 50 nM and 1 uM. For example, A83-01 may be added to the culture medium at approximately 500 nM. Other useful TGF-β type I receptor inhibitors include, but are not limited to SB431542 (4-[4-(1,3)-benzodioxol-5-yl)-5-(2-pyridinyl)-1H-imidazol-2 yl]benzamide); LY 364947 (4-[3-(2-Pyridinyl)-1H-pyrazol-4-yl]-quinoline); R 268712 (4-[2-Fluoro-5 [3-(6-methyl-2-pyridinyl)-1/1pyrazol-4-yl]phenyl]-1H-pyrazole-1-ethanol); SB 525334 (6-[2-(1,1-Dimethylethyl)-5-(6-methyl-2-pyridinyl)-1H-imidazol-4-yl]quinoxaline); and SB 505124 (2-[4-(1,3-Benzodioxol-5-yl)-2-(1,1-dimethylethyl)-17 imidazol-5-yl]-6-methyl-pyridine)

Y-27632 (thins-4-[(1R)—I-Aminoethyl]-2%-4-pyridinylcyclohexanecarboxamide dihydrochloride) is a selective p160ROCK inhibitor. Other useful Rho inhibitors include isoquinolin and (S)-(+)-2-methyl-1-[(4-methyl-5-isoquinolinyl)sulfonyl]-hexahydro-1H-1,4-diazepine dihydrochloride (H-1152; Tocris Bioscience).

In particularly preferred embodiments, the AO or PD cell culture media used in the disclosed methods includes an ErbB3/4 ligand (e.g. human neuregulin β-1). The ErbB receptor tyrosine kinase family consists of four cell surface receptors, ErbB1/EGFR HER1, ii) ErbB2/HER2, iii) ErbB3/HER3, and iv) ErbB4/HER4. ErbB3/4 ligands include members of the neuregulin/heregulin family. The neuregulin % heregulin family is referred to herein as the neuregulin family. The neuregulin family is a family of structurally related polypeptide growth factors that are gene products of alternatively spliced genes NRG1, NRG2, NRG3 and NRG4. In more preferred embodiments, the excluded one or more ErbB3/4 ligands of the culture medium are polypeptides that are gene products of one or more of NRG1, NRG-2, NRG3 and NRG4 {i.e. a neuregulin polypeptide).

(iii). PD Culture Medium

A preferred PD medium is a cell culture medium suitable for air-liquid interface culture of bronchial epithelial cells. In some embodiments, the PD medium comprises one or more (or all) of the components listed in Table 2, preferably at the concentrations shown in Table 2.

TABLE 2
Composition of PD medium.

| PD medium components | Exemplary concentrations | |
|---|---|---|
| Basal medium | 50:50 mixed LHC basal medium and | |
| | DMEM medium supplemented with | |
| | retinoic acid (50 nM) | |
| EGF | 0.5 | ng/ml |
| bovine serum albumin | 150 | mg/ml |
| bovine pituitary extract | 10 | ug/ml |
| insulin | 5 | ug/ml |
| transferrin | 10 | ug/ml |
| hydrocortisone | 72 | ng/ml |
| triiodothyronine | 6.7 | ng/ml |
| epinephrine | 0.6 | ug/ml |

| | |
|---|---|
| antibiotics | Penicillin-Streptomycin (100 U/ml), |
| | Gentamicin (50 ug/ml) and/or |
| | Amphotericin B (0.25 ug/ml) |

In some embodiments, the PD medium is serum free and/or BPE (bovine pituitary extract)-free. An example of a suitable PD medium is the commercially available PneumaCult-ALI medium (StemCell Technologies). PneumaCult™-ALI Medium is a serum- and BPE-free medium for the culture of human airway epithelial cells at the air-liquid interface (ALI). Airway epithelial cells cultured in PneumaCult™-ALI Medium undergo extensive mucociliary differentiation to form a pseudostratified epithelium that exhibits morphological and functional characteristics similar to those of the human airway in vivo. PneumaCult™-ALI Medium supports the generation of differentiated airway organoids in a 2D or 3D culture system.

In a particularly preferred embodiment, the PD medium is supplemented with a notch inhibitor, preferably in a concentration range between 5 and 30 μM, preferably between 10 and 20 μM and more preferably about 10 μM.

Examples of preferred Notch inhibitors that can be used in the context of this invention are: gamma-secretase inhibitors, such as DAPT or dibenzazepine (DBZ) or benzodiazepine (BZ) or LY-411575, an inhibitor capable of diminishing ligand mediated activation of Notch (for example via a dominant negative ligand of Notch or via a dominant negative Notch or via an antibody capable of at least in part blocking the interacting between a Notch ligand and Notch), or an inhibitor of ADAM proteases. In a particularly preferred embodiment, the notch inhibitor is DAPT ([N—(N-[3,5-difluorophenacetyl]-L-alanyl)-S-phenylglycine t-butyl ester).

The isolated cells cultured in AO medium are subsequently cultured in PD medium for a period of time effective for formation of PD-organoids. In one preferred embodiment, the time period of time effective for formation of PD-organoids is from about five to about 20 days. In another preferred embodiment, the period of time effective for formation of airway organoids is about 14 days.

B. 2D PD Organoids

2D PD organoids may be obtained from 3D airway organoids by a method that includes dissociating the 3D AO into a single cell suspension, seeding the cells in transwell inserts and culturing the cells in AO medium followed by culture in PD medium for a period of time effective for formation of an intact epithelial barrier, as measured for example, by a dextran penetration assay. The 3D organoids are dissociated into single cells using methods known in the art (discussed herein), preferably, by enzymatic dissociation, followed by shearing and straining over a filter as disclosed in the Examples.

The dissociated cells are cultured as a monolayer, preferably on a permeable support (cell culture insert) in AO medium at 37° C. in a humidified incubator with 5% CO₂ for 1-2 days and then cultured in PD medium as a monolayer for a time period between 5 and 16 days, preferably between 10 and 14 days, and more preferably, for about 12-14 days to obtain 2D PD-organoids. The PD medium is preferably provided on the apical and basolateral sides of the monolayer. Permeable supports are commercially available, for example, Corning® Transwell®. Transwell inserts are convenient, ready-to-use permeable support devices pre-packaged in standard multiple well plates. The unique, self-centered hanging design prevents medium wicking between the insert and outer well. Transwell inserts are available in a wide variety of sizes, membrane types, and configurations.

### IV. Methods of Using the Composition

The disclosed 3D and 2D proximal differentiated airway organoids can morphologically and functionally simulate human airway epithelium.

Organoids derived from adult stem and progenitor cells reliably retain their in vivo regenerative activity in vitro, and thus provide detailed snapshots of tissue restoration after injury. Lung organoids allow researchers to study processes governing homeostatic regulation of lung tissue and screen factors that impact lineage-specification of stem cells.

Accordingly, the disclosed PD-organoids may be used as an alternative to live animal testing for compound or for treatment of (including resistance to treatment of) lung infection or disease (e.g., chronic obstructive pulmonary disease (COPD)).

Influenza virus infection represents a major threat to public health worldwide. The disclosed 3D and 2D proximal differentiated airway organoids can morphologically and functionally simulate human airway epithelium and can discriminate human infective influenza viruses from poorly infective viruses. Thus, the proximal differentiated airway organoids can be utilized to determine the infectivity of influenza viruses and significantly extend advances in influenza research and provide solutions to influenza infection. One of the most important and challenging issues for infectious disease research, for example, influenza research is to predict which animal or emerging influenza virus can infect humans. In one embodiment, a method for determining infectivity of a pathogen for example a non-human strain of the influenza virus in humans, by comparing infectivity of the non-human virus in the disclosed 3D or 2D differentiated airway organoids, and comparing its infectivity with a strain of that pathogen known to be highly infectious in humans (high infectivity control) and a strain of that pathogen known have no or low infectivity in humans (low-infectivity control). For example, human infective H7N9/Ah and H1N1pdm can be used as positive control and H7N2 or swine H1N1 used as negative control to determine compare their replication in the 2D or 3D organoids compared to the virus whose infectivity in humans is being tested. Replication in the 2D or 3D organoid comparable with H7N9/Ah and H1N1pdm, indicates that the virus being tested would be infective in humans. Conversely, replication comparable to H7N2 or swine H1N1 indicates that the virus being tested would be low infectivity in humans.

For acute treatment testing, compound or vaccine may be applied to the PD-organoid, e.g., once for several hours. For chronic treatment testing, compound or vaccine may be applied, e.g., for days to one week. Such testing may be carried out by providing an airway organoid product as described herein under conditions which maintain constituent cells of that product alive (e.g., in a culture media with oxygenation); applying a compound to be tested (e.g., a drug candidate) to the lung PD-organoid (e.g., by topical or vapor application to the epithelial layer); and then detecting a physiological response (e.g., damage, infection, cell proliferation, cell death, marker release such as histamine release, cytokine release, changes in gene expression, etc.), the presence of such a physiological response indicating said compound or vaccine has therapeutic efficacy, toxicity, or other metabolic or physiological activity if inhaled or otherwise delivered into the airway of a mammalian subject. A control sample of the PD-organoid may be maintained under like conditions, to which a control compound (e.g., physiological saline, compound vehicle or carrier) may be applied, so that a comparative result is achieved, or damage can be determined based on comparison to historic data, or comparison to data obtained by application of dilute levels of the test compound, etc.

In some preferred embodiment, the disclosed PD-organoid is can be used for influenza virus testing (infectivity and vaccines). In a particularly preferred embodiment, the disclosed PD-organoid can discriminate human infective influenza viruses from poorly infective viruses. Thus, the proximal differentiated airway organoids can be utilized to predict the infectivity of influenza viruses and significantly extend the current armamentaria of influenza research toolbox.

Pre-clinical models of human disease are essential for the basic understanding of disease pathology and its translational application into efficient treatment for patients. Patient-derived organoid cultures from biopsies and/or surgical resections can be used for personalized medicine. Two examples are lung cancer and cystic fibrosis. Additionally, tissue samples can be obtained from a subject cultured as disclosed herein and used to determine the subject's responsiveness to medication in order to select the better treatment for that subject. Dekkers et al. Science Translational Medicine, 8(344):344ra84 (2016) showed that the efficacy of cystic fibrosis transmembrane conductance regulator (CFTR)-modulating drugs can be individually assessed in a laboratory test using epithelial cells cultured as mini-guts from rectal biopsies from subjects with cystic fibrosis. The authors show that the drug responses observed in mini-guts or rectal organoids can be used to predict which patients may be potential responders to the drug. Similar preclinical tests using the disclosed 3D organoids obtained from a subject may help to quickly identify responders to CFTR-modulating drug therapy even when patients carry very rare CFTR mutations.

Ex vivo expanded adult stem cell-derived organoids retain their organ identity and genome stability, and can be differentiated to PD lung organoids as described herein. Therefore, the PD-organoids may also be used for replacing damaged tissues.

Airway organoids can easily be established from bronchiolar lavage material of humans, allowing inter-individual comparisons; airway organoids can also be readily modified by lentiviruses and CRISPR technologies and can be single cell-cloned. In combination with the molecular toolbox of influenza virologist, the human differentiated airway organoid model system offers great opportunities for studying virus and host factors that define characteristics of this major animal and human pathogen.

The present invention will be further understood by reference to the following non-limiting examples.

### V. Examples

A. Materials and Methods

Establishing Adult Stem Cell-Derived Human Airway Organoids.

Generation of adult stem cells (ASC) derived human airway organoids was based on the following protocol. Briefly, upon ethical approval by Institutional Review Board of the University of Hong Kong/Hospital Authority Hong Kong West Cluster (HKU/HA HKW IRB, UW 13-364) and informed consents of patients, small pieces of normal lung tissues around 0.5-0.8 cm in size and adjacent to the diseased tissues, were obtained from patients who underwent surgical operation. The tissues were minced and digested with 2 mg/ml collagenase (Sigma Aldrich) for 1-2 hours at 37° C., followed by shearing using glass Pasteur pipette and straining over a 100 μm filter. The resultant single cells were embedded in 60% MATRIGEL™ and were seeded in 24-well suspension culture plate. After solidification, MATRIGEL™ droplets containing single cells were maintained with airway organoid (AO) culture medium (Table 1) at 37° C. in a humidified incubator with 5% CO₂. The organoids were passaged every 2-3 weeks. The bright field images of the organoids were acquired using Nikon Eclipse TS100 Inverted Routine Microscope. To generate PD organoids, airway organoids were split and cultured in AO medium for 2-7 days, following which the culture medium was changed to PD medium.

Proximal Differentiation of Human Airway Organoids.

The airway organoids were split and maintained in AO medium for 2-7 days. The culture media in half of the organoids were changed to proximal differentiation (PD) medium, PneumaCult-ALi medium (StemCell Technologies) supplemented with 10 μM DAPT, a notch pathway inhibitor (Tocris). The organoids were then cultured AO or PD media for 16 days, to obtain 3D AO-airway organoids and 3D-PD airway organoids, respectively. Bright field images were taken every 3 days. Diameters of individual organoids were measured with ImageJ. The movies of organoids were acquired using Total Internal Reflection Fluorescent (TIRF) Microscope and Nikon Eclipse Ti2 Inverted Microscope System. At the indicated days, the organoids in the different media were harvested for detection of cellular gene expression or applied to flow cytometry analysis.

Establishing 2D Differentiated Airway Organoids with Transwell Culture.

Transwell culture of airway organoids was performed as described elsewhere (24, 25) with modifications. Briefly, the 3D airway organoids were dissociated into single cell suspension after digested with 10×TrypLE™ Select Enzyme (Invitrogen) for 1˜5 min at 37° C., sheared using Pasteur pipette and strained over a 40 μm filter. Approximately 3.5×10⁵ cells were seeded into each transwell insert (Corning, product #3494). The cells were cultured in AO medium at 37° C. in a humidified incubator with 5% CO₂ for 1-2 days. When cells reached >90% confluence, the AO medium was changed to PD medium in both the apical and basal chambers. The medium was changed every other day and the cells were maintained for 14 days. Trans-epithelial electronic resistance (TEER) was measured every other day using Millicell ERS-2 Volt-Ohm Meter (EMD Millipore). To assess the integrity of the 2D organoid monolayer as an epithelial barrier, at day 12 after seeding, fluorescein isothiocyanate-dextran with an average molecular weight of 10,000 (Sigma Aldrich) was added in the medium of upper chamber at a concentration of 1 mg/ml and incubated at 37° C. for 4 hours. Subsequently, the culture media were harvested from the upper and bottom chambers to detect the fluorescence intensity using the Victor XIII Multilabel Reader (PerkinElmer).

Propagation of Influenza a Viruses.

Influenza A virus A/Anhui/1/2013(H7N9) (H7N9/Ah), A/Hong Kong/415742/2009(H1N1) (H1N1pdm) and swine H1N1 isolate (H1Nsw) were propagated in Madin-Darby Canine Kidney (MOCK) cells. At 72 hours post inoculation (hpi), cell-free medium was harvested, aliquoted and stored at −80° C. Avian IAVs H7N2 and Viet Nam/1194/04 (H5N1) was propagated in special pathogen-free embryonated chicken eggs at 37° C. for 36 hours. The eggs were chilled for overnight at 4° C.; then the virus-containing allantoic fluid was harvested, aliquoted and stored at −80° C. Virus titer was determined by plaque assay.

Influenza a Virus Infection in Human Airway Organoids.

The 3D airway organoids were sheared mechanically to expose the apical surface to the virus inoculum. The sheared organoids were then incubated with viruses at a multiplicity of infection (MOI) of 0.01for 2 hours at 37° C. After washing, the inoculated organoids were re-embedded into MATRIGEL™ and then cultured in the PD medium. In the H7N9/Ah and H7N2 infection in the 3D PD organoids, one set of H7N9/Ah-inoculated organoids were treated with a serine proteases inhibitor AEBSF (0.125 mM, Merck Millipore) during inoculation and after inoculation. At the indicated hpi, organoids, dissolved MATRIGEL™ and culture medium were harvested for detection of viral load. The cell-free MATRIGEL™ and the culture medium from each droplet were pooled together as one sample, referred as supernatant. The supernatant samples were also used for viral titration. The 2D PD airway organoids were inoculated with the indicated viruses at an MOI of 0.001, from the apical side by adding the virus inoculum into the apical chamber and incubating for 2 hours at 37° C. At the indicated hpi, cell-free media were collected from apical and basolateral chambers for subsequent viral titration. The membranes seeded with 2D organoids were incised from transwell inserts, fixed and applied to immunofluorescence staining as described previously (13).

RNA Extraction, Reverse Transcription and Quantitative Polymerase Chain Reaction (RT-qPCR).

To evaluate the differentiation status of airway organoids cultured in PD medium versus those in AO medium, the organoids were harvested at the indicated hours and applied to RNA extraction using MiniBEST Universal RNA extraction kit (TaKaRa). To evaluate virus replication, the organoids and supernatant samples were lysed for RNA extraction using MiniBEST Universal RNA extraction kit and MiniBEST Viral RNA/DNA Extraction Kit (TaKaRa) respectively. Complementary DNA (cDNA) was synthesized with Transcriptor First Strand cDNA Synthesis Kit (Roche) with Oligo-dT primer. qPCR was performed with LightCycler 480 SYBR Green I Master (Roche) using gene specific primers (Table 3) to detect cellular gene expression level and viral gene copy number. The mRNA expression levels of cellular genes were normalized with that of GAPDH. Viral gene copy number was determined by absolute quantification using a plasmid expressing a conserved region of IAV M gene.

TABLE 3
Primers for quantitative PCR assay.

| Gene Name | Primer Sequence | |
|---|---|---|
| p63 (TP63) | F | CAGACTCAATTTAGTGAGCC (SEQ |
| | | ID NO: 1) |
| | R | CTGCTGGTCCATGCTGTT (SEQ ID |
| | | NO: 2) |
| keratin 5 (KRT5) | F | GAGGAATGCAGACTCAGTGGA |
| | | (SEQ ID NO: 3) |
| | R | TAGCTTCCACTGCTACCTCCG (SEQ |
| | | ID NO: 4) |
| forkhead box J1 | F | TCGTATGCCACGCTCATCTG (SEQ |
| (FOXJ1) | | ID NO: 5) |
| | R | CGGATTGAATTCTGCCAGGT (SEQ |
| | | ID NO: 6) |
| sentan, cilia | F | GCTGCAAACCCAATTTAGGA (SEQ |
| apical structure | | ID NO: 7) |
| protein (SNTN)* | R | TGCTCATCAAGTTCAGAAAGGA |
| | | (SEQ ID NO: 8) |
| mucin 5AC, | F | CCTACAAAGCTGAGGCCTGT (SEQ |
| oligomeric | | ID NO: 9) |
| mucus/gel- | R | GACCCTCCTCTCAATGGTGC (SEQ |
| forming | | ID NO: 10) |
| (MUC5AC) | | |
| secretoglobin | F | AGCATCATTAAGCTCATGGAAAAA |
| family 1A | | (SEQ ID NO: 11) |
| member 1 | R | GTGGACTCAAAGCATGGCAG (SEQ |
| (SCGB1A1) | | ID NO: 12) |
| secretoglobin | F | AACTGCTGGAGGCGCTATCA (SEQ |
| family 3A | | ID NO: 13) |
| member 2 | R | TGTCCTTTTCACGGGTCACT (SEQ |
| (SCGB3A2) | | ID NO: 14) |
| transmembrane | F | CTTTGAACTCAGGGTCACCA (SEQ |
| protease, serine 2 | | ID NO: 15) |
| (TMPRSS2) | R | TAGTACTGAGCCGGATGCAC (SEQ |
| | | ID NO: 16) |
| transmembrane | F | TGCTTCAGGAAACATACCGA (SEQ |
| protease, serine 4 | | ID NO: 17) |
| (TMPRSS4) | R | CTGGAGTGAGCTCCTCATCA (SEQ |
| | | ID NO: 18) |
| transmembrane | F | TACACAGGAATACAGGACTT (SEQ |
| protease, serine | | ID NO: 19) |
| 11D | R | CTCACACCACTACCATCT (SEQ ID |
| (TMPRSS11D) | | NO: 20) |
| Matriptase | F | CTAGGATGAGCAGCTGTGGA (SEQ |
| | | ID NO: 21) |
| | R | AAGAATTTGAAGCGCACCTT (SEQ |
| | | ID NO: 22) |
| IAV M gene | F | CTTCTAACCGAGGTCGAAACG |
| | | (SEQ ID NO: 23) |
| | R | GGCATTTTGGACAAAKCGTCTA |
| | | (SEQ ID NO: 24) |

Plaque Assay.

Plaque assay was performed to determine titers of the virus stocks and supernatant samples as described elsewhere with minor modification (26). Briefly, MDCK cells were seeded in 12-well plates. Confluent monolayers were inoculated with 200 μL of 10-fold serial dilutions of samples and incubated for 1 hour at 37° C. After removing the inoculum, the monolayers were overlaid with 1% LMP Agarose (Invitrogen) supplemented with MEM and 1 μg/μ1 TPCK-treated Trypsin and further incubated for 2-3 days. The monolayers were fixed with 4% PFA and stained with 1% crystal violet to visualize the plaque after removing the agarose plugs. Virus titers were calculated as plaque-forming units (PFU) per milliliter.

Immunofluorescence Staining

To identify the indicated cell types and the virus-infected cells, the 3D and 2D airway organoids were applied to immunofluorescence staining. Briefly, the organoids fixed with 4% PFA, permeabilized with 0.1-5% Triton X-100 and blocked with Protein block (Dako). Then the organoids were incubated with primary antibodies (Table 4) diluted in Antibody Diluent buffer (Dako) overnight at 4° C., followed by incubation with secondary antibody (Table 4) for 1˜2 hours at room temperature. Nuclei and actin filaments were counterstained with 4′-6-diamino-2-phenylindole (DAPI) (Invitrogen) and Phalloidin-647 (Sigma Aldrich) respectively. The confocal images were acquired using Carl Zeiss LSM 780 or 800.

TABLE 4
List of Antibodies for used for incubation.

| Antibodies | Company | Catalog No. |
|---|---|---|
| Mouse Anti-Cytokeratin 5 | Abcam | ab128190 |
| Rabbit Anti-p63 | Abcam | ab124762 |
| Mouse Anti-β-tubulin 4 | Sigma | T7941 |
| Mouse Anti-FOX J1 | Invitrogen | 14-9965-82 |
| Mouse Anti-Mucin 5AC | Abcam | ab3649 |
| Rat Anti-Uteroglobin/CC-10 | R&D Systems | MAB4218-SP |
| Rabbit Anti-Influenza A NP | Novus | NBP2-16965 |
| Goat Anti-Mouse, Alexa Fluor 488 | Invitrogen | A11001 |
| Goat Anti-Mouse Alexa Fluor 594 | Invitrogen | A11005 |
| Goat Anti-Rabbit Alexa Fluor 488 | Invitrogen | A11034 |
| Goat Anti-Rabbit Alexa Fluor 594 | Invitrogen | A11037 |
| Goat Anti-Rat Alexa Fluor 594 | Invitrogen | A11007 |

Flow Cytometry Analysis.

To assess the percentage of four types of cells, the airway organoids were applied to flow cytometry analysis. Briefly, the organoids were dissociated with 10 mM EDTA (Invitrogen) for 30˜60 minutes at 37° C., fixed with 4% PFA and permeabilized with 0.1% Triton-100. Subsequently, the cells were incubated with primary antibodies (Table 4) for 1 hour at 4° C. and followed by secondary antibodies staining. BD FACSCantoII Analyzer was used to analyze the samples.

Statistical Analysis

Student's t test was used for data analysis. P<0.05 was considered to be statistically significant.

B. Results

Characterization of the Human Airway Organoids.

Several lines of airway organoids (3D cysts lined by polarized epithelium) were established as discussed briefly above, using the OA culture medium, the lung cell culture medium disclosed in U.S. Published Application No. 2017/275592. The four main types of airway epithelial cells were present, i.e. ciliated cell (ACCTUB+ or FOXJ1+), basal cell (P63+), goblet cell (MUC5AC+), and Club cell (CC10+). Apical ACCTUB clearly indicated the orientation of polarization. Most organoids were orientated inwards the lumen; while a small proportion of the organoids were inverted. Beating cilia were visible. No type I and type II alveolar epithelial cells was present. Thus, these organoids resembled the pseudostratified ciliated airway epithelium. The airway organoids were infected by human IAV H1N1pdm, low pathogenic avian virus H7N9/Ah and highly pathogenic avian virus H5N1 (FIGS. 1A-1C). The intracellular (cell lysate) viral loads of all 3 virus strains increased over 2 log₁₀ units (FIG. 1A). The extracellular (supernatant) viral loads (FIG. 1B), especially the viral titers (FIG. 1C), were elevated by 2-3 log₁₀ units.

Ciliary beating plays essential roles in human airway biology and pathology, and 50%-80% of airway epithelial cells are ciliated (Yaghi, et al., Cells, 5(4): pii:E402016)). However, by immunostaining and flow cytometry, ciliated cells were apparently under-represented in these airway organoids. Therefore, despite the discernible multi-lineage differentiation and the ability to support replication of IAVs, further improvement of morphology and differentiation appeared required. Furthermore, when these AO-organoids are passaged over time, less and less cilia can be observed. After consecutively passaging 3 months, cilia are not detectable.

Proximal Differentiation of the Airway Organoids.

To improve proximal differentiation, various protocols and variations thereof were investigated, selecting a proximal differentiation (PD) medium supplemented with DAPT ([N—(N-[3,5-difluorophenacetyl]-L-alanyl)-S-phenylglycine t-butyl ester to induce ciliary differentiation. The organoids in the original airway organoid (AO) medium gradually enlarged, whilst those in PD medium became more compact. After 16 days of culture, the organoids in AO medium grew 2 times larger approximately, while the PD organoids basically remained unchanged (FIG. 2). From day 7, numbers of ciliated cells increased markedly in PD medium. At day 16, beating cilia were observed in a minority of (<10%) the organoids in AO medium, whilst abundant beating cilia were present in every PD organoid. The synchronously beating cilia drove the cell debris within the organoid lumens to swirl unidirectionally. The dramatically increased abundance of ciliated cells in the PD organoids was verified by immunofluorescence staining. This is in contrast to the 3D organoids obtained from LBO (lung bud organoids) disclosed in Chen, et al., Nat Cell Biol 19(5):542-549, (2017), where in vitro cultures are strongly biased towards distal lung, and, although some areas co-expressing SOX2 and SOX9 expressed more proximal markers for goblet cells and club cell precursors, mature club cells, ciliated cells or basal cells were not observed. Nikolic, et al., Elife 6: e26575 (2017))

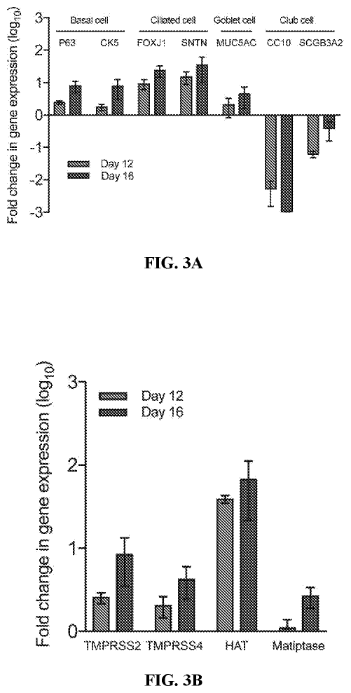

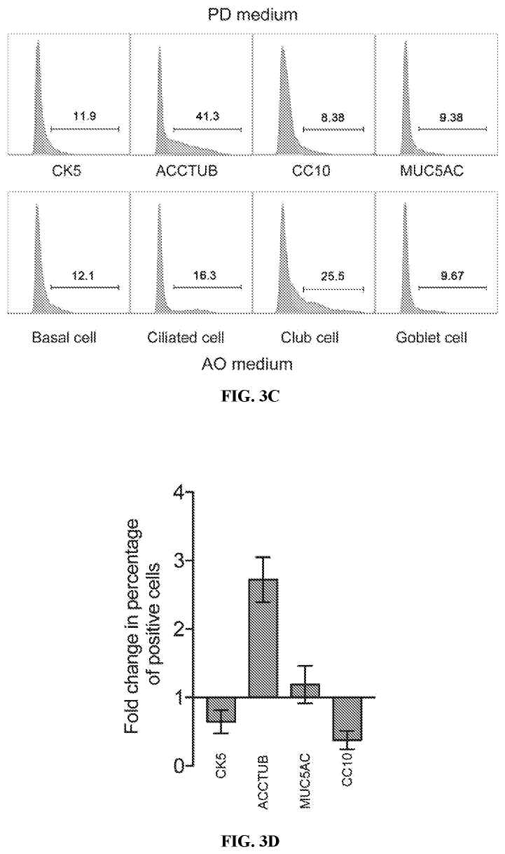

Consistently, the transcriptional levels of ciliated cell markers, FOXJ1 and SNTN, were strongly upregulated in the PD organoids compared with the organoids in AO medium. The expression levels of basal cell markers (P63, CK5) and goblet cell marker (MUC5AC) also increased; whereas the levels of Club cell markers (CC10, SCGB3A2) were substantially downregulated in the PD organoids (FIGS. 3A and 3B). Importantly, globally elevated expression of serine proteases including TMPRSS2, TMPRSS4, TMPRSS11D (HAT) and Matriptase was observed, which are essential for the activation and propagation of human IAVs and low pathogenic avian IAVs (8). Flow cytometry analysis was also performed to measure the percentages of the four cell types in the organoids cultured in two distinct media at day 16. It was shown that, the percentage of ciliated cell remarkably increased around 3-fold after proximal differentiation, to over 40% in the PD organoids; while the ciliated cells invariably constituted a minority of the cells in the organoids in AO medium (FIGS. 3C and 3D). Goblet cells also marginally increased; while Club cells consistently decreased after proximal differentiation (FIGS. 3C and 3D). Collectively, mucociliary differentiation and developed proximal differentiated airway organoids which can morphologically and functionally simulate human airway epithelium was successfully induced in the original airway organoids.

Proximal Differentiated Airway Organoids can Identify Human Infective Virus.

One of the most important and challenging issues for influenza research is to predict which animal or emerging influenza virus can infect humans. As mentioned above, the novel reassortant avian H7N9 viruses have caused continuing poultry-to-human transmission since 2013. Other subtypes of avian IAVs (including H7N2, H9N2 and H9N9) have been co-circulating with the H7N9 viruses in domestic poultry. These viruses are highly similar in internal genes; yet differ in neuraminidase (NA) or HA and NA (18). Very few human infections by H7N2, H9N2 and H9N9 virus have been reported in the same territory and time frame although people were exposed to these viruses equivalently as to the H7N9 viruses (19), suggesting that these viruses are less-infective to humans than the H7N9 viruses.

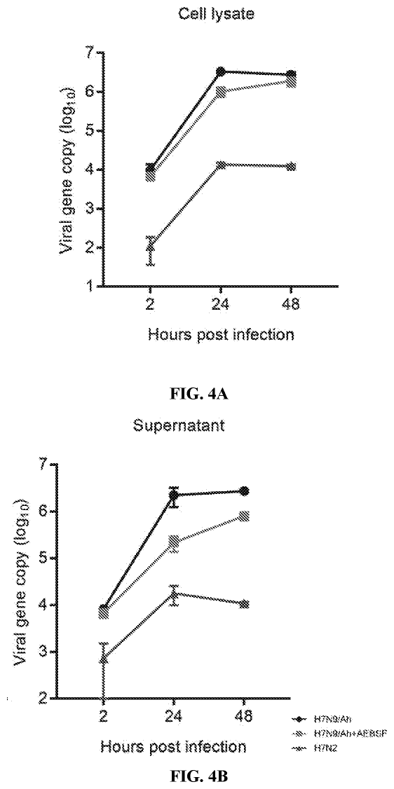

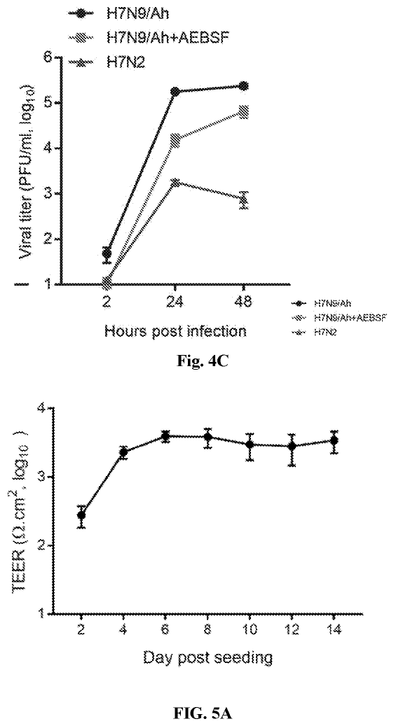

These co-circulating viruses were isolated, plaque purified and genotyped. H7N2 and H7N9/Ah was chosen to compare their infectivity in the PD organoids, with the hypothesis that the differentiated airway organoids can indeed simulate human airway epithelium in the context of influenza virus infection. FIG. 4A-C showed that viral loads in the cell lysate and medium of H7N9/Ah-infected organoids gradually increased after inoculation; the viral titer increased more than 3 log₁₀ units within 24 hours, indicating a robust viral replication. The addition of serine protease inhibitor AEBSF significantly restricted the active replication of H7N9/Ah virus, highlighting the importance of elevated serine proteases for viral replication. In contrast, H7N2 modestly propagated with viral titer 2-3 log₁₀ units lower than H7N9/Ah. Thus, the distinct efficiency of H7N9/Ah and H7N2 to infect and replicate in proximal differentiated airway organoids can recapitulate infectivity of these viruses in humans.

Establishing 2D Airway Monolayer from Airway Organoids to Assess the Infectivity of IAVs.

3D organoids were transformed into a 2D monolayer using transwell culture. To this end, 3D airway organoids were enzymatically dissociated into single cell suspension, seeded in transwell inserts and then cultured in PD medium. The trans-epithelial electronic resistance (TEER) in the 2D monolayers stabilized in the second week after seeding (FIGS. 5A and 5B). In addition, the dextran penetration assay performed at day 12 indicated that an intact epithelial barrier was formed cross the 2D monolayers (FIGS. 5A and 5B). The intense signal of ACCTUB indicated that the 2D monolayers developed appreciable proximal differentiation. The productive infection of H7N9/Ah was clearly shown by the virus nucleoprotein (NP) positive cells at 8 hours post infection (hpi).

The replication capacity of H7N9/Ah and H7N2 in the 2D PD organoids was compared. To further verify the ability of 2D PD organoids for assessing zoonotic potential of animal viruses, and identifying the human-infective virus, the replication capacity of H7N9/Ah and H7N2 in the 2D PD organoids, as well as another pair of viruses, the highly human infective H1N1pdm and a swine H1N1 isolate (H1N1sw) were analyzed. The higher replication capacity of H7N9/Ah over H7N2 virus was more pronounced in the 2D PD organoids than in the 3D PD organoids; the viral titer of H7N9/Ah in the apical media was 3-4 log₁₀ units higher than that of the H7N2 virus (FIGS. 5C and 5D). Consistently, H1N1pdm dramatically replicated with viral titer in apical media 1-2 log₁₀ units higher than H1N1sw. Due to the epithelial barrier formed in the 2D monolayers and the preferential virus release from the cell apical side, the viral titers in the basolateral media were consistently lower than those in apical media at the corresponding time points. Nevertheless, the differences in replication capacity between H7N9/Ah versus H7N2, H1N1pdm versus H1N1sw were even more prominent in basal media than in apical media in most time points.

C. Discussion

This study describes proximal differentiation of human ASC-derived airway organoid culture for studies of a major pathogen, the influenza virus. In particular, the disclosed differentiation conditions increase the numbers of ciliated cells (FIGS. 3A and 3C), and the major cell type in the human airway epithelium. The PD medium induces ciliated cell numbers to a near-physiological level, with synchronously beating cilia readily discernible in every organoid. In addition, the expression levels of serine proteases (FIG. 3B) known to be important for productive viral infection, were elevated after proximal differentiation. Among the upregulated HA-activating serine proteases, the dramatically increased expression of HAT in the differentiated airway organoids is very likely attributed to the increased abundance of ciliated cells since ciliated cells are the main source of HAT in the human respiratory tract (Krueger, et al., Swine Influenza Virus Infections in Man. Swine Influenza, eds Richt JA & Webby RJ (Springer Berlin Heidelberg, Berlin, Heidelberg), pp 201-225 (2013)). Thus, the differentiated airway organoids can morphologically and functionally simulate the human airway epithelium. As a further improvement, 2D PD airway monolayers were developed, with an intact epithelial barrier to allow exclusive apical exposure to viruses (FIGS. 5A and 5B), the natural mode of IAV infection in the human respiratory tract. Two pairs of viruses with known infectivity were utilized to demonstrate, as a proof-of-concept, that these organoids indeed show significantly higher susceptibility to the human-infective viruses than the poorly human-infective viruses. These 3D and 2D differentiated airway organoids support active replication of human infective H7N9/Ah and H1N1pdm. In contrast, the H7N2 virus, which has been temporally and spatially co-circulating with H7N9 viruses in domestic poultry and contains the similar internal genes as H7N9 viruses, replicated much less efficiently in both models. Similarly, the swine H1N1 isolate showed a lower growth capacity than its counterpart of human-adapted H1N1pdm (FIGS. 5C and 5D).

The avian IAV H7N2 subtype viruses circulating in the bird market between 1994-2006 caused poultry outbreaks in the US. Sporadic human infections have been reported in the US and Europe. Fortunately, all reported human infection cases experienced mild influenza-like symptoms (Marinova-Petkova, et al., Emerg Infect Dis 23(12) (2017)). While pigs are considered to be the intermediate hosts for interspecies transmission of IAVs, swine influenza viruses lacking human adaptation markers rarely infect humans. Sporadic human infections documented in the literatures or reported by public health officials are generally mild or subclinical (Krueger, et al., Swine Influenza Virus Infections in Man. Swine Influenza, eds Richt JA & Webby RJ (Springer Berlin Heidelberg, Berlin, Heidelberg), pp 201-225 (2013)). The ability of the PD airway organoids to differentiate avian H7 subtype virus and swine H1 subtype virus from the counterpart human viruses suggest that these models could be used for assessment of cross-species transmission potential of emerging influenza virus in humans.

In summary, these differentiated airway organoids significantly extend the current armamentaria of influenza research toolbox.

Unless defined otherwise, all technical and scientific terms used herein have the same meanings as commonly understood by one of skill in the art to which the disclosed invention belongs. Publications cited herein and the materials for which they are cited are specifically incorporated by reference. Those skilled in the art will recognize, or be able to ascertain using no more than routine experimentation, many equivalents to the specific embodiments of the invention described herein. Such equivalents are intended to be encompassed by the following claims.

1. A method of generating a proximal differentiated airway organoid (PD-organoid) comprising culturing an airway organoid (AO-organoid) in a proximal differentiation medium for a period of time sufficient to generate a PD-organoid comprising a cell population consisting of at least 25%, at least 30%, at least 35% or at least 40% ciliated cells, wherein the ciliated cells are characterised by FOXJ1 and SNTN expression.

2. The method of claim 1, wherein the proximal differentiation medium is supplemented with a notch inhibitor, optionally selected from the group consisting of a gamma-secretase inhibitor, such as DAPT or dibenzazepine (DBZ) or benzodiazepine (BZ) or LY-411575.

3. (canceled)

4. The method of claim 2, wherein the notch inhibitor is DAPT, preferably at a concentration of between 5 and 30 μM, preferably between 10 and 20 μM, or more preferably about 10 μM.

5. The method of claim 1, wherein the proximal differentiation medium comprises one or more components as set out in Table 2, optionally at the concentrations shown in Table 2; and/or wherein the proximal differentiation medium is PneumaCult-ALI medium (StemCell Technologies) supplemented with notch inhibitor.

6. The method of claim 5, wherein the proximal differentiation medium comprises at least EGF, insulin, transferrin, hydrocortisone, triiodothyronine and epinephrine.

7. The method of claim 6, wherein the proximal differentiation medium further comprises bovine serum albumin and/or bovine pituitary extract.

8. (canceled)

9. The method of any claim 1, wherein the method further comprises one or more of the following steps prior to culturing the AO-organoid in a proximal differentiation medium:
a. obtaining a lung tissue sample from a subject;
b. obtaining dissociated cells from a lung tissue sample; and
c. culturing lung cells in an AO-organoid formation phase for a period of time sufficient to generate an AO-organoid.

10. The method of claim 9, wherein the AO-organoid formation phase comprises culturing cells in an AO-organoid medium comprising one or more components as set out in Table 1, optionally at the concentrations shown in Table 1.

11. The method of claim 10, wherein the AO-organoid medium comprises at least R-spondin, a BMP inhibitor, a TGF-beta inhibitor, FGF and heregulin beta-1.

12. The method of claim 11, wherein the step of culturing the lung cells and/or AO-organoid comprises culturing the cells in contact with an exogenous extracellular matrix (such as a basement membrane extract or Matrigel™).

13. The method of claim 1, wherein: (a) the AO-organoid is a 3D organoid; (b) the PD-organoid is a 3D organoid; and/or (c) the PD-organoid is a 2D organoid.

14. (canceled)

15. (canceled)

16. The method of claim 13, wherein the step of culturing in a proximal differentiation medium comprises culturing in a transwell culture system comprising an apical and basal chamber.

17. A method of generating a 3D PD-organoid in accordance with claim 13 comprising the steps of:
a. culturing lung cells from a subject in an AO-organoid formation phase in an AO-organoid medium in contact with an extracellular matrix for a period of time sufficient to generate a 3D AO-organoid, for example for at least 2 days; and
b. changing the AO medium to a proximal differentiation medium supplemented with a notch inhibitor and culturing the 3D AO-organoid in the proximal differentiation medium supplemented with a notch inhibitor for a period of time sufficient to generate a PD-organoid, for example for at least 5 days, at least 10 days, at least 14 days or at least 16 days.

18. A method of generating a 2D PD-organoid in accordance with claim 13 comprising the steps of:
a. culturing lung cells from a subject in an AO-organoid formation phase in an AO-organoid medium in contact with an extracellular matrix for a period of time sufficient to generate a 3D AO-organoid, for example for at least 2 days;
b. dissociating the 3D AO-organoids into single cell suspension;
c. seeding the dissociated cells in the apical chamber of a transwell culture system;
d. optionally culturing the seeded cells in AO medium for at least 1 day, for example, until the cells reach at least 90% confluence; and
e. culturing the seeded cells in proximal differentiation medium supplemented with a notch inhibitor for a period of time sufficient to generate a 2D PD-organoid, for example for at least 5 days, at least 10 days, at least 14 days or at least 16 days.

19. The method of claim 16, wherein: (a) the culture medium is added to both the apical and basal chambers of the transwell culture system; (b) wherein the culture medium is refreshed every other day; and/or (c) the organoid or cells are human organoids or human cells.

20. (canceled)

21. (canceled)

22. A PD-organoid obtained by a method of claim 1, wherein the PD-organoid consists of a cell population comprising at least 25%, at least 30%, at least 35% or at least 40% ciliated cells, wherein the ciliated cells are characterised by FOXJ1 and SNTN expression.

23. The PD-organoid of claim 22, wherein: (a) the PD-organoid has at least 2-fold or at least 3-fold increase in the proportion of ciliated cells when compared to the AO-organoid from which it is derived; (b) the PD is further characterised by serine protease expression, for example, expression of one or more or all of TMPRSS2, TMPRSS4, TMPRSS11D (HAT) and Matriptase; (c) expression of HAT is at least 1 log₁₀ fold increased relative to its expression in AO-organoids; and/or (d) the ciliated cells make up at least 10-40% of the cells in the organoid by day 12, by day 14, or by day 16 after culturing in the proximal differentiation medium.

24. (canceled)

25. (canceled)

26. (canceled)

27. The PD-organoid of claim 22, further comprising one or more or all of the following cell types:
a. basal cells, characterised by P63 and CK5 expression;
b. goblet cells, characterised by MUC5AC expression; and
c. club cells characterised by lack of CC10 and SCGB3A2 expression.

28. The PD-organoid of claim 22, wherein gene expression is assessed using quantitative PCR of mRNA transcripts normalised with GAPDH; and/or (b) the PD-organoid further comprises an influenza virus.

29. (canceled)

30. (canceled)

31. A method for contracting an influenza virus in a PD-organoid, wherein the method comprises:
a. generating a PD-organoid in accordance with claim 1; and
b. infecting the PD-organoid with an influenza virus.

32. The method of claim 31, wherein: (a) the infecting step comprises inoculating with the influenza virus at a multiplicity of infection of at least 0.001, at least 0.01 or between 0.001 and 0.01; (b) the infecting step further comprising incubating for at least 30 minutes, at least 60 minutes, at least 90 minutes or at least 120 minutes; (c) the contacting step is at the apical surface of the PD-organoid; (c) the PD-organoid is a 2D organoid and contacting step involves adding the influenza virus to the apical chamber of the transwell culture system or (d) the PD-organoid is a 3D organoid and the method further comprises a step of exposing the apical surface of the 3D organoid, for example by mechanical shearing, prior to contacting the PD-organoid with an influenza virus.

33. (canceled)

34. The method of claim 32, wherein the incubating step is performed at about 37° C.; or the method further comprises re-contacting the 3D organoid with an extracellular matrix and culturing the PD-organoid in a proximal differentiation medium, after infecting, and optionally incubating, the PD-organoid with the influenza virus.

35. (canceled)

36. (canceled)

37. (canceled)

38. (canceled)

39. A method for predicting infectivity of a test influenza virus to humans, wherein the method comprises:
a. generating a human PD-organoid in accordance with claim 1;
b. contacting the human PD-organoid with the test influenza virus;
c. testing the viral titre after a time period sufficient to allow viral propagation;
d. optionally comparing the viral titre to a control influenza virus.

40. The method of claim 39, wherein: (a) testing the viral titre involves detecting a change in viral titre; (b) the control influenza virus is a known poorly-infective-to-humans influenza virus, optionally wherein the change in viral titre of the test influenza virus is greater than the change in viral titre of the known poorly-infective-to-humans influenza virus, for example wherein the viral titre is at least 10-fold, at least 50-fold, at least 100-fold, at least 1,000 fold or at least 10,000 fold greater than the viral titre of the known poorly-infective-to-humans influenza virus; or (c) the control influenza virus is a known infective-to-humans influenza virus, optionally wherein the change viral titre of the test influenza virus is about the same or greater than the viral titre of the known infective-to-humans influenza virus, for example, at least 75%, at least 80%, at least 90%, at least 100%, at least 150%, at least 2-fold, at least 5-fold or at least 10-fold relative to the viral titre of the known infective-to-humans influenza virus.

41. The method of claim 40, wherein an increase in viral titre is indicative of likely infectivity of the influenza virus to humans and/or wherein a greater increase over a shorter time period is correlated with a higher degree of infectivity and optionally, wherein the increase in viral titre is at least 1 login units, at least 2 log₁₀ units, or at least 3 log₁₀ units within 24 hours.

42. (canceled)

43. (canceled)

44. The method of claim 41, wherein the known poorly-infective influenza virus is selected from H7N2, H9N2 and H9N9.

45. (canceled)

46. (canceled)

47. The method or PD-organoid of claim 1, wherein the influenza virus is:
a. an influenza A virus;
b. a human, avian or swine influenza virus; and/or
c. an emerging influenza virus.
